(19) United States
(12) Patent Application Publication
(10) Pub. No.: US 2017/0299581 A1
(43) Pub. Date: Oct. 19, 2017

(54) ELECTROCHEMICAL TYROSINASE ENZYME IMMUNOASSAY BIOSENSOR AND SYSTEMS AND METHODS RELATED THERETO

(71) Applicant: BOARD OF TRUSTEES OF MICHIGAN STATE UNIVERSITY, East Lansing, MI (US)

(72) Inventors: Robert M. WORDEN, Holt, MI (US); Paul S. SATOH, Holt, MI (US)

(21) Appl. No.: 15/383,320

(22) Filed: Dec. 19, 2016

Related U.S. Application Data
(60) Provisional application No. 62/268,878, filed on Dec. 17, 2015.

(51) Int. Cl. G01N 33/543; G01N 33/94; G01N 27/30; C12Q 1/00; G01N 27/327; G01N 27/49; G01N 33/569

(52) U.S. Cl. CPC G01N 33/5438; G01N 27/49; C12Y 114/18001; C12Y 301/03001; G01N 33/948; G01N 33/56911; C12Q 1/005; G01N 27/3271; G01N 27/308; G01N 2333/28; G01N 2333/90245; G01N 2333/916

(57) ABSTRACT

Devices, assays and methods for detecting analytes in a sample are provided. Biosensor devices include a biosensor interface that includes enzyme-conjugated molecules, antibodies and an enzyme driven redox cycle coupled to an electrically conductive electrode for signal amplification. The biosensor devices are easily adaptable to a variety of assay formats, a variety of target analytes and provide real-time measurements combined with high sensitivity and high specificity for the analyte.

This application claims the benefit under 35 U.S.C. 119(e) of U.S. Provisional Application Ser. No. 62/268,878 filed on Dec. 17, 2015.

### STATEMENT OF GOVERNMENT RIGHTS

This invention was made with government support under IIP1444991 awarded by the National Science Foundation. The government has certain rights in the invention.

### BACKGROUND

Biosensor devices that include an electrically conductive electrode and a biosensor interface that is attached to a surface of the electrode, wherein the interface includes catalytically active material that facilitates electron transfer, has been shown. The catalytically active material may be directly or indirectly bound to the electrically conductive electrode. Bioelectronic interfaces achieve electrical communication between redox enzymes and electrodes.

### SUMMARY

The embodiments described herein provide devices, assays and methods for detecting analytes in a sample. In particular, the various embodiments provide biosensor devices comprising enzyme conjugated molecules and a biosensor interface that includes an enzyme driven redox cycle and molecules that recognize an analyte, such as immunological molecules, coupled to an electrically conductive electrode for signal amplification.

Advantages of using the biosensor devices of the present disclosure include, but are not limited to, economical production of biosensors that are versatile and can easily be configured to a variety of target analytes. The biosensor can detect virtually any analyte for which antibodies can be produced. The biosensors can be used for real-time measurements (within several seconds) combined with high sensitivity even at low analyte concentrations and also high specificity for the analyte. Further, the present description relates to biosensor devices that can be used in medicine, industrial chemistry, agricultural uses and homeland security. The electrochemical biosensors described herein can provide a more quantitative measurement than the commonly used antibody-based optical assays.

In the present disclosure, the advantages of the redox cycling signal amplification mechanism are combined with the extremely high binding affinity and selectivity of antibodies. In various embodiments, the biosensor can use a biosensor interface coupled to an electrode in combination with generating a “sandwich” structure when a target analyte is present. By analogy, with a sandwich, the primary analyte binding material or primary antibody can be immobilized in or on the biosensor interface and is analogous to the bottom layer of a sandwich. A liquid sample, potentially containing an analyte, can then be exposed to the biosensor interface. If molecules of the target analyte are present in the sample, they can bind to the primary antibodies to form the middle layer of the sandwich. The sample solution can then be replaced with another composition containing secondary analyte binding material, e.g. secondary antibodies, that can be attached to a reporter enzyme such as a hydrolase, as the term is defined herein, which includes, but is not limited to, various types of esterases (e.g., lipases and phosphatases (including nucleases), glycosidases and peptidases.

In various embodiments, the reporter enzyme can be conjugated to secondary antibodies which can bind to the immobilized analyte molecules, thus forming the top layer of the sandwich. The composition with the secondary analyte binding material can, in one embodiment, be then replaced by a substrate composition containing a substrate that, when acted upon by the reporter enzyme, produces a product that is a trigger molecule. The trigger molecule can trigger a reaction in a redox cycle amplification pathway, leading to generation of an electric current if the target analyte is present in the sample. In one embodiment, the amount of current generated can correlate with the amount of the target analyte in the sample.

In various embodiments, the biosensor interface can include a variety of enzymes, including one or more enzymes that drive a redox cycle within the biosensor device for amplification of the target analyte signal. For example, a hydrolase enzyme conjugated to the secondary analyte binding material can convert a substrate into a product, such as phenol or other phenolic compounds which can trigger the redox cycling loop. The phenolic compound may, in one embodiment, be oxidized by an enzyme of the redox cycle to a quinone. In such an embodiment, the quinone can then be reduced back to generate a phenolic compound, e.g., catechol, by electron transfer from the electrode. The phenolic compound can, in one embodiment, be re-oxidized by enzyme(s) propagating the redox cycling loop between one or more quinones, e.g., o-quinone, and one or more phenolic compound, e.g., catechol. In other words, the regeneration of the oxidized and reduced forms of the electroactive molecule, e.g. a quinone, by the biosensor device can propagate the redox cycle. In one embodiment, the current generated by the electrochemical reduction of the quinone constitutes the biosensor's electrical output.

In this sandwich assay format embodiment, the biosensor interface initially contains no reporter enzyme, e.g. a hydrolase, so the biosensor current produced is essentially zero prior to exposure to the target analyte. When the biosensor interface is exposed to a sample containing the target analyte, the analyte can, in various embodiments, bind to the primary antibodies. Then, when the secondary antibodies conjugated to a reporter enzyme are added, they can bind the analyte which is already bound to the primary antibody, thus creating a sandwich architecture that binds the reporter enzyme to the biosensor interface. Finally, in one embodiment, addition of the substrate triggers the redox cycle, resulting in a biosensor current.

Based on this mechanism, in one embodiment, the higher the analyte concentration in the sample, the higher are the analyte and secondary antibody loadings, and the higher the resulting biosensor signal. Because the initial background signal from the biosensor is essentially zero prior to analyte addition in various embodiments, even a small initial analyte concentration can provide a relatively large signal-to-background noise ratio. Thus, one advantage of the sandwich assay configuration is a relatively high sensitivity for detecting low analyte concentrations.

The lower detection limit can vary from system to system. As the analyte concentration drops, the signal can drop. At some concentration, the signal can become so low that it can be indistinguishable from the noise. This concentration can help define the lower detection limit of that biosensor for its analyte.

In various embodiments, a competitive-displacement assay format may be used. In this assay format embodiment, a sample containing the analyte can be contacted with the biosensor interface that can include the redox enzyme and the primary analyte binding material. The biosensor interface can be pre-saturated, before the addition of a test sample, with hybrid reporter enzyme/analyte molecules in one embodiment, which include reporter enzyme that can be attached to analyte molecules. When a sample is added to the biosensor interface, analyte molecules in the sample can displace a fraction of the hybrid reporter enzyme/analyte molecules bound to the primary antibodies, thereby reducing the reporter enzyme concentration in the interface and reducing the biosensor's current output. Thereafter, in one embodiment, the current level can be measured and used to determine the analyte concentration in the sample. In one embodiment of the competitive-displacement assay, an increase in the amount of analyte in a sample correlates with a decrease in the signal from the biosensor device.

In various embodiments, the biosensor devices may be compatible with a lateral flow assay, a vertical flow assay and a printed electrode such as a screen-printed electrode.

### BRIEF DESCRIPTION OF THE FIGURES

FIG. 1 is a schematic of an electrochemical enzyme immunoassay biosensor of the present disclosure in accordance with an embodiment.

FIG. 2A is a schematic of the molecular architecture of an electrochemical enzyme immunoassay biosensor interface architecture with a tyrosinase enzyme in the redox cycling and signal amplification in accordance with an embodiment.

FIG. 2B is a schematic of the underlying chemical reactions of an electrochemical enzyme immunoassay biosensor interface architecture with a tyrosinase enzyme for redox cycling and signal amplification in accordance with an embodiment.

FIGS. 3A and FIG. 3B are schematics of competitive displacement assays of the present disclosure in accordance with various embodiments.

### DESCRIPTION OF THE EMBODIMENTS

In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized, and structural, logical, mechanical, electrical, and other changes may be made.

Various terms are defined herein. See also definitions in U.S. Pat. No. 8,623,196 to Kohli et al. (hereinafter the '196 patent), U.S. Pat. No. 8,435,773 to Worden et al. (hereinafter the '773 patent) and U.S. patent application Ser. No. 12/766,169 by Worden et al. (hereinafter the '169 application), all of which are hereby incorporated herein by reference in their entireties. In case of a conflict in the meaning of various terms, the definitions provided herein should prevail.

The singular forms of the terms “a”, “an”, and “the” as used herein include plural references unless the context clearly dictates otherwise. For example, the term “a biosensor” includes a plurality of biosensors.

The term “enzyme” as used herein refers to a molecule that acts as a catalyst for a chemical reaction, such that a substrate is converted into a product.

The terms “Enzyme Commission number” or “E.C. number” or “EC number” or “E.C.” or “EC” as used herein refer to a numerical classification scheme for enzymes. Each EC number refers to a specific enzyme-catalyzed reaction under one number and a generic name, however each number may include one enzyme or several different enzymes.

The terms “substrate” or “reactant” as used herein refer to a substance that is acted upon by an enzyme during a chemical reaction. This includes, but is not limited to, alkaline phosphatase (ALP) acting upon phenyl phosphate substrate to form phenol as the product.

The term “product” as used herein refers to a substance that forms as a result of a chemical reaction. The product can be the product of a chemical reaction catalyzed by an enzyme.

The term “trigger molecule” as used herein refers to a substance that directly or indirectly triggers the activation of the redox cycle. By indirectly, it is meant that the trigger molecule may not be part of the redox cycle but may be acted upon by an enzyme such that the product of that reaction is a component of the redox cycle loop. In other words, the trigger molecule can trigger the initial activation of the redox cycle but may not necessarily be involved within the redox loop cycle.

The term “reporter enzyme” as used herein refers to an enzyme conjugated to the secondary analyte binding material, the analyte or other molecules. Reporter enzyme can bind a substrate and catalyze a reaction wherein the product of the reaction is a trigger molecule that can trigger the redox cycle directly or indirectly.

The term “hybrid reporter enzyme/analyte molecules” as used herein refers to a reporter enzyme conjugated to an analyte.

The terms “phenolic molecule” and “reduced phenolic molecule” as used herein refer to various oxidative states of phenol and include both the intermediate reduced state, e.g. catechol, and the most reduced state, e.g. phenol.

The term “quinone” as used herein refers to the most oxidized state of a phenolic molecule, e.g. o-quinone.

The term “o-quinone” as used herein refers to ortho-quinone.

The terms “oxidation-reduction” or “oxidation/reduction” or “redox” or “re-dox” as used herein, when used in reference to a reaction, refer to the transfer of electrons between molecules, such that molecules may gain or lose electrons to become “electroactive species.”

The term “oxidation” as used herein refers to the loss of electrons.

The term “reduction” as used herein refers to the gain of electrons.

The term “redox cycle” as used herein refers to the chemical reactions that cycle molecules between their oxidized states and reduced states. Components in the redox cycle can include, but are not limited to, redox enzymes, reducing agents, oxidizing agents, oxidized molecules, reduced molecules and other molecules that are necessary to drive the redox cycle such as oxygen, hydrogen peroxide (H₂O₂), catalysts (enzymatic and non-enzymatic), conductive materials, such as nanoparticles that increase the effective surface area of the electrode, and the like.

The term “redox enzymes” as used herein refers to protein-based molecules, e.g., enzymes that catalyze oxidation and reduction reactions and are involved in driving the redox cycle. Enzymes of the redox cycle amplification pathway can also include, but are not limited to, enzymes that may not be directly involved in the redox cycle but provide components and/or catalyze reactions that enable the redox cycle to be initiated and/or propagated.

The terms “oxidizing agent” or “oxidant” as used herein refer to a substance that affects oxidation by accepting electrons from another substance or adding oxygen to another substance.

The terms “reducing agent” or “reductant” as used herein refer to a substance that affects reduction by donating electrons to another substance or removing oxygen from another substance.

The terms “conjugation” or “conjugate” or “conjugated to” and the like, as used herein refer to formation of a stable linkage between a biomolecule and another molecule. Conjugation can include formation of a covalent bond between two biomolecules, such as an enzyme and an antibody. Conjugation can also include a covalent bond between an enzyme and an analyte.

The terms “tyrosinase enzyme” or “tyrosinase” as used herein, when used in reference to a protein, refer to a tyrosinase protein (monophenol monooxygenase) (EC 1.14.18.1) that catalyzes the oxidation of phenols or phenolic compounds (such as phenol, catechol, and the like). Tyrosinase also refers to a molecule with a systemic name of monophenol, L-dopa: oxygen oxidoreductase in addition to a molecule with any one of the following names: phenolase; monophenol oxidase; cresolase; catechol oxidase; polyphenolase; pyrocatechol oxidase; dopa oxidase; chlorogenic oxidase; catecholase; polyphenol oxidase; monophenolase; o-diphenol oxidase; chlorogenic acid oxidase; diphenol oxidase; o-diphenolase; tyrosine-dopa oxidase, o-diphenol:oxygen oxidoreductase; polyaromatic oxidase; monophenol monooxidase; o-diphenol oxidoreductase; monophenol dihydroxyphenylalanine:oxygen oxidoreductase; N-acetyl-6-hydroxytryptophan oxidase; monophenol, dihydroxy-L-phenylalanine oxygen oxidoreductase; o-diphenol:O₂oxidoreductase; phenol oxidase, and the like.

The terms “alkaline phosphatase” or “alkaline phosphomonoesterase” or “phosphomonoesterase” or “EC 3.1.3.1” as used herein refer to an enzyme for catalyzing a reaction comprising a phosphate monoester and H₂O to form substrates of an alcohol and a phosphate.

The terms “esterase” or “esterase enzyme” as used herein refer to an enzyme, such as a protein, that when functional may catalyze the hydrolysis of an ester or a synthetic substrate comprising an ester group. In other words, an esterase refers to a hydrolase enzyme that when functional may split an ester into an acid and an alcohol in a chemical reaction with water, also referred to as E.C. 3.1 an enzyme that acts on an ester bond, and includes, but are not limited to, enzymes such as carboxylic ester hydrolases (EC 3.1.1); lysophospholipase (EC 3.1.1.5); acetyl esterase (EC 3.1.1.6); acetylcholinesterase (EC 3.1.1.7); and cholinesterase (EC 3.1.1.8).

The terms “Neuropathy target esterase” or “NEST” as used herein, when used in reference to a protein, refer to proteins that are identical to a wild-type neuropathy target esterase or a portion thereof.

The terms “acetylcholinesterase” or “AchE” as used herein refer to a protein that catalyzes the hydrolysis of acetic esters, such as serine esters, acetic esters in acetylcholine, and catalyzes transacetylations, also designated EC 3.1.1.7. Acetylcholinesterase also refers to a molecule with a systemic name of acetylcholine acetylhydrolase, true cholinesterase; choline esterase I; cholinesterase; acetylthiocholinesterase; acetylcholine hydrolase; acetyl β-methylcholinesterase; AcCholE, and the like.

The terms “butylcholinesterase” or “butyrylcholinesterase” or “BChE” or “BuChE” or “pseudocholinesterase” or “plasma cholinesterase” or “liver cholinesterase” as used herein refer to an enzyme, also designated EC 3.1.1.8; that catalyzes the hydrolysis of the neurotransmitter acetylcholine into choline and acetic acid.

The term “hydrolase” as used herein refers to an enzyme (e.g. esterase, phosphatase, glycosidase and peptidase) that can catalyze the hydrolysis of various types of compounds. Esterases (E.C. 3.1) include, but are not limited to, lipases, which break ester bonds (between a carboxylic acid and an alcohol) in lipids, and phosphatases (including nucleases), which act analogously upon phosphates. Glycosidases (E.C.3.2) sever bonds between sugar molecules in carbohydrates. Peptidases (E.C.3.40 hydrolyze peptide bonds (between the carboxylic acid group of one amino acid and the amino group of another) within protein molecules.

The term “nuclease” as used herein refers to a phosphatase that hydrolyzes nucleic acids.

The term “oxidase” as used herein refers to an enzyme that catalyzes oxidation. The enzyme can react with molecular oxygen as the electron acceptor to catalyze the oxidation of a substrate.

The term “analyte” as used herein refers to any chemical compound, biomolecule, bacteria, virus or portions thereof susceptible to immobilization assays including immunoassays and polynucleotide hybridization assays. Analytes are generally pathogens, toxins or other microorganisms or molecules for which detection is desired. Examples of analytes include, but are not limited to, the fish pathogen, e.g. Vibrio harveyi, tetrahydrocannabinol (THC), cortisol, and the like.

The term “target analyte” as used herein refers to the analyte of interest in a sample to be detected or characterized.

The term “sample” as used herein refers to a composition that may or may not include an analyte. A sample may be either aqueous, dissolved in an organic solvent or a solid sample that may be dissolved in either water, aqueous solution or an organic solvent. A sample is meant to include a liquid or solid or gas. A sample is meant to include, but not be limited to, biological samples, environmental samples, chemical samples, and the like. The samples may be applied to the biosensor described herein. The biosensor may also be immersed into a sample.

The terms “primary analyte binding material” or “capture analyte binding material” as used herein refer to any of a variety of compounds and biomolecules used in immobilization assays to bind one or more analytes to a surface. These include, but are not limited to, primary antibodies, lipid layers, and protein binding materials such as styrene, polynucleotides and combinations thereof.

The terms “primary antibody” or “capture antibody” as used herein refer to an antibody comprising a primary analyte binding material configured to bind the target analyte.

The term “secondary analyte binding material” as used herein refers to any material configured to bind to an analyte and being conjugated to a reporter enzyme. This includes, but is not limited to, secondary antibodies as used in ELISA's and polynucleotide probes used in hybridization assays

The term “secondary antibody” as used herein refers to an antibody comprising a secondary analyte binding material configured to bind the target analyte and being conjugated to a reporter enzyme.

The term “assay structure” as used herein refers to any structure suitable for performing the electrochemical detection and immobilization assays described herein. This includes any of the assay structures disclosed ranging from centimeter scale to nanometer scale devices. An assay structure comprises at least one surface to which an analyte binds and at least one electrode for detection of current.

The term “immunoassay” as used herein measures the presence and/or concentration of an analyte using immunological molecules in the test. The analyte can be large proteins or small molecules. Immunoassays can be applied to or compatible with many formats. Immunoassays are designed to detect the binding of the specific antibody to the target analyte.

The term “specificity” as used herein refers to the characteristic inherent to various molecules that allows them to recognize and bind to a specific molecule. For example, all antibodies are specific for a certain antigen. Also, any single stranded polynucleotide has specificity for a complementary strand because it will not anneal to a non-complementary strand. The specificity of antibodies, DNA probes, aptamers, enzymes and substrates enable chemical detection assays. Non-antibody protein-based molecules that can be designed to bind with high affinity to a target analyte molecules may also be used.

The term “nanostructured” as used herein refers to a device having at least one surface or coating of which the physical and chemical properties or features are in the nanometer range or smaller.

The term “response time” as used herein refers to the time it takes for a sensor's output to reach some fraction of its final value. The response time is a measure of how quickly the sensor will respond to changes in the environment. In general, this parameter is a measure of the speed of the sensor and must be compared with the speed of the process.

The term “sensitivity” as used herein refers to an amount of change in a sensor's output in response to a change at a sensor's input over the sensors entire range. The sensitivity provides an indication of a sensor's ability to detect changes. For some sensors, the sensitivity is defined as the input parameter change required to produce a standardized output change.

The terms “signal-to-noise-ratio” or “signal-to-background noise ratio” as used herein refer to a ratio of the output signal with an input signal to the output signal with no input signal,

The terms “subject” or “patient” as used herein refer to any animal, such as any type of mammal, including, but not limited to a dog, cat, bird, livestock, or human.

The term “operably linked” as used herein refers to the attachment of amino acid sequences or polypeptides to other molecules or surfaces in such a manner so that the function of the protein is preserved. The attachment can be covalent, electrostatic, entrapment, and the like.

The terms “bacteria” and “bacterium” as used herein refer to all prokaryotic organisms, including those within the phyla in the Kingdom Prokaryote. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive.

The term “microorganism” as used herein refers to a microscopic organism and taxonomically related macroscopic organisms within the categories of algae, bacteria, fungi (including lichens), Archaea, protozoa, viruses, and subviral agents.

The terms “attach” or “attached” as used herein refer to attaching molecules to each other and attaching molecules to the surface of a device refers to the adherence of one molecule to another molecule or the adherence of molecules to an inorganic surface. For the purposes of the present description attaching a molecule to an electrode may occur directly or indirectly. For example, a sensor-coupling; molecule may be attached directly to an inorganic surface of an electrode whereas an enzyme may be attached indirectly to an electrode by attaching the enzyme to other molecules that in turn are attached to the electrode. Attachment of molecules to each other and molecules to inorganic surfaces may be accomplished by a variety of methods, such as by forming covalent bonds, electrostatic forces, entrapment, and the like.

The term “immobilizing” as used herein refers to attaching a molecule or macromolecule so it is stationary or unable to diffuse away, i.e., immobilized. Immobilization may occur through carrier binding, cross-linking, entrapping, and the like.

The term “enzyme immobilizing agent” as used herein refers to a substance that attaches an enzyme to a biosensor of the present description. Examples of an enzyme immobilizing compound include, but are not limited to, any covalent bond forming compound, such as a cross-linking compound (e.g., via glutaraldehyde, thioctic acid, and the like), an entrapment compound, (e.g., polyacrylamide), a charged compound that can attached to the enzyme via ionic interactions (e.g., a positively charged polyelectrolyte, and the like, such as polylysine), a sensor-coupling molecule, and the like.

The term “substance” as used herein refers to a compound or agent or carrier or molecule.

The term “covalently attached” as used herein refers to the attachment of at least two moieties by at least one covalent bond.

The term “covalent bond forming compound” as used herein refers to a molecule forming a chemical bond that results in the sharing of one or more pairs of electrons (e.g. amide bonds), such as a cross-linking compound, a linker molecule, a spacer molecule, an entrapment compound, a sensor-coupling molecule, and the like.

The term “entrapment” as used herein refers to attaching a protein to a biosensor interface surface. Entrapment can use a compound, molecule or polymer to hold a protein in place. The protein can be held by a polymer matrix such as polyacrylamide, polyelectrolytes such as polylysine, polystyrene, and the like. Entrapment may also include use of porous materials to retain the protein or enzymes. The protein can bind, for example, to a polyelectrolyte of the opposite charge by, but not limited to, ionic interactions.

The term “biosensor interface” as used herein refers to the area encompassing the organic portion of a biosensor in contact with the inorganic electrode surface. A biosensor interface, in reference to an organic portion of a biosensor, refers to the area where the organic portion is in contact with a test sample. A biosensor interface can include, but is not limited to, redox cycle components, primary and/or secondary analyte binding materials, reporter enzymes and other organic components.

The term “bioelectronic interface” as used herein refers to the organic and inorganic components of the biosensor device of an assay system that involves one or more electron transfer process or redox reaction.

The term “sensor-coupling” as used herein refers to a substance (such as a compound, agent, carrier, molecule, coating, and the like) which acts to bridge organic and inorganic molecules. When used to bridge organic and inorganic surfaces, it is referred to as a “spacer molecule.” Such sensor-coupling molecules form stable bonds, such as covalent bonds, between organic and inorganic molecules. Examples of coupling molecules include, but are not limited to, thioctic acid, thioctic acid derivatives, such as 2-aminoethyl-D-mannopyranoside; 2-aminoethyl-1,3-D-mannopyranosyl(-1,6-D-mannopyranosyl)-D-mannopyranoside, thiourea, 3-mercaptopropionic acid, and the like, organosilicon compounds, silanes, such as amino, epoxy, acrylate, methacrylate, mercapto, vinyl silanes, such as 3-Acryloxypropyl)trimethoxysilane, N-(2-Aminoethyl)-3-aminopropyltrimethoxysilane, 3-Aminopropyltriethoxysilane, 3-Aminopropyltrimethoxysilane, 3-Isocyanatopropyltriethoxysilane, 3-Glycidoxypropyl)trimethoxysilane, 3-Mere aptopropyltrimethoxysilane, 3-Methacryloxypropyltrimethoxysilane, Vinyltrimethoxysilane (Gelest, Inc.) silanes, and the like. It is to be understood that a sensor-coupling molecule can be used herein to attach an organic enzyme to an inorganic electrode surface.

The term “biosensor” as used herein refers to a general designation that encompasses a sensor. A biosensor, in reference to a measuring device, refers to a biological interface (for example, an enzyme, cell layer, etc.) coupled to a transducer (for example, a gold electrode, an oxygen-type electrode, a carbon composite electrode, and the like). It is to be understood that the term biosensor, in reference to an electrode, refers to an analytical device for converting a biological response into an electrical signal, such as an amperometric, calorimetric, potentiometric, optical, or piezo-electric signal, and the like The biosensor may be used in a laboratory setting, or may be embedded in a large reservoir, such as a reactor, or in a subject's body for in-situ use.

The term “amperometric” as used herein refers to the measurement of an electric current flowing under an applied electrical potential difference between two electrodes in contact with an electrolyte. When used in reference to an electrochemical analysis, amperometric refers to a detected or measured current which, in turn, varies with the concentration of the species generating the current. When used in reference to an electrode, such as “amperometric electrode” or “amperometric biosensor,” amperometric refers to an electrode in which electron transfer occurs at the surface of an electrode (e.g., working electrode) and generates a current.

The term “amperometric detection” as used herein refers to a method of applying a voltage or electric potential difference to the electrode containing the compositions of the present description (the working electrode) and the reference electrode (in a two-electrode cell) or the auxiliary electrode (in a three-electrode cell).

The term “analytical information” as used herein refers to measurements obtained from a current-concentration relationship at a specified applied voltage.

The term “electrolyte” as used herein refers to a liquid or any form of matter that provides a path for ion flow, such as a solution that is configured to conduct an electric current.

The term “polysalt” as used herein refers to a polymer whose repeating unit bears an electrolyte group, including, but not limited to, a polyacid, a polybase, a polysalt, a polyampholyte, a polylysine, a polyacrylic acid, and the like.

The term “polyelectrolyte” as used herein refers to a polymer whose repeating unit bears an electrolyte group including, but not limited to, polylysine, polyethylenimine, and diethylaminoethyl-dextran and poly(amidoamine).

The term “electrode” as used herein refers to an electrically conductive surface that is part of a biosensor. The biosensor is connected to an electronic device or instrumentation which applies any necessary voltage between the electrodes of the biosensor and measures any current, resistance, and the like that represent the biosensor's signal. The signal is then used to infer, for example, the concentration of an analyte in the analyte binding sites of the working electrode (biosensor interface). For the purposes of the present description, the term “working electrode” refers to an electrode that serves as a transducer responding to an excitation signal, such as an electrode of the present description. For the purposes of the present description, the term “reference electrode” refers to an electrode used to measure the relative electrical potential of a different electrode, such as a “silver/silver chloride” or “Ag/AgCl” reference electrode.

The terms “counter electrode” or “auxiliary electrode” as used herein refer to an electrode used to make a connection to the electrolyte so that a current can be applied to the working electrode. The counter electrode is usually made of an inert material, such as a noble metal, for example, a metal or alloy, such as gold or graphite.

The term “electrode potential” as used herein refers to a difference in electrical potential between an electrode and a reference electrode in contact with an electrolyte. For example, the working electrode on which the enzymes are bound, and where o-quinone might be reduced to catechol, can be maintained at a given potential relative to a reference electrode (e.g., −0.1V relative to a Ag/AgCl reference electrode).

The terms “voltage” or “volt” or “V” as used herein refer to the difference of electrical potential between two points of an electrical or electronic circuit, measured as a unit of electrical potential difference or volt.

The term “voltammetry” as used herein refers to an electrochemical measuring technique used for electrochemical analysis or for the determination of the kinetics and mechanism of electrode reactions. “Voltammetry” also refers to family of techniques with a common characteristic where a potential of the working electrode is controlled (for example, with a potentiostat) and the current flowing through the electrode is measured, for example, potential step voltammetry, linear sweep voltammetry, cyclic voltammetry, AC voltammetry, and the like.

The term “potential step voltammetry” as used herein refers to a measurement technique involving an applied voltage that is instantaneously jumped from one value (V1) to another value (V2). The values of V1 and V2 can vary depending on the objectives of the experiment.

Electrochemical biosensors that measure a protein's activity have been widely used to determine analyte concentrations, both in research and commercial applications. Biosensor devices can detect protein activity either directly or indirectly through reaction coupling. The indirect approach offers the potential to greatly broaden the range of protein classes that can be characterized using electrochemical biosensors. Redox enzymes, such as oxidase and dehydrogenase enzymes, are well suited for electrochemical characterization because the oxidation and reduction reactions they catalyze can result in generation of an electric current that can be monitored using electrodes.

General immunoassay procedures can involve immobilization of a molecule or macromolecule (e.g., protein). In one embodiment, general immunoassay procedures can involve immobilization of a primary antibody (i.e., capture antibody) followed by exposure to a sequence of solutions containing the analyte, and a secondary antibody conjugated to an enzyme label. The use of chemical compounds specific to the analyte such as antibodies further increases the possibilities of specifically detecting an analyte in a sample. Enzyme-linked immunosorbent assay (ELISA) systems combine the high binding specificity of antibodies with signal amplification provided by the linked enzyme. In ELISA systems, enzymes rapidly form products that can be detected and quantitated by optical detection methods. The virtually unlimited number of reaction cycles the enzyme can undergo allows order-of-magnitude amplification of the optical signal, allowing ELISA systems to have extremely low detection thresholds.

In various embodiments described herein, biosensor devices for assays, assay structures and methods for detecting an analyte are disclosed. In one embodiment, the biosensor device is a nanostructured biosensor device on the order of about 10⁻⁹ meters or smaller (<10⁻⁹ meters). In one embodiment, the biosensor device is larger than a nanostructured biosensor device and is on the order of about 10⁻⁹ meters or greater (>10⁻⁹ meters

The biosensor devices of the present description can increase biosensor sensitivity by integrating multiple mechanisms of signal amplification, including, but not limited to, enzyme-antibody linkage that translates antibody-analyte binding events into a cascade of chemical reactions and a redox cycle that converts chemical reactions into an electric current and dramatically amplifies the current. Conductive nanomaterials that can massively increase the electrode surface area and reaction density may also be incorporated into the devices. A broad range of analytes can be measured by extending the redox-cycling mechanism to other enzymes, enzyme-linked antibodies and enzyme-linked analytes.

The resulting bioelectronic interfaces of the devices can be configured to a variety of biosensor platforms such as disposable biosensors used for rapid, point-of-care assays, electrode-containing multiwell plates used for high-throughput applications, and flow injection analysis (FIA) systems widely used in clinical and environmental laboratories. In each of these platforms, the biosensor interfaces can provide sensitive, quantitative electrochemical assays for a diverse range of analytes.

In various embodiments, the working electrode used with a reference electrode allows the working electrode's potential to be set at a value that carries out the desired oxidation or reduction reaction. In one embodiment, the working electrode is used with a reference electrode and an auxiliary electrode that allows the working electrode's potential to be maintained at a constant value even when an oxidation or reduction reaction is occurring. In various embodiments, the oxidation or reduction reaction generates a current, whose magnitude (amperage) provides a measure of the reaction rate. The measurement of current generated at the working electrode is known amperometric detection.

In various embodiments, a current is measured at a chosen applied voltage of a working electrode with respect to a reference electrode for obtaining analytical information from the current-concentration relationship at that applied voltage. In one embodiment, the current obtained or measured depends on the concentration of an analyte such that the analyte causes an oxidation/reduction of an electro-active species at the surface of a working electrode.

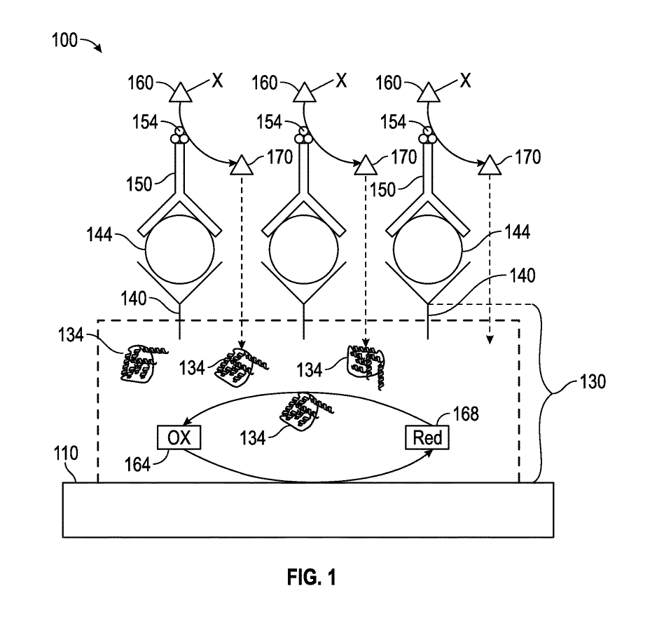

FIG. 1 shows a schematic diagram of one embodiment of a sandwich structure biosensor device in an assay system 100 of the present description, which can include electrically conductive electrode 110 attached to biosensor interface 130. In one embodiment, electrode 110 is a gold electrode. In one embodiment, electrode 110 is a carbon electrode. Electrode 110 can also include conductive particles such as carbon nanoparticles in contact with electrode 110 as extensions that increase electrode 110's effective surface area. Electrode 110 may also include other types of electrodes and optionally, other conductive particles known in the art and all are within the scope of the present disclosure.

In various embodiments, biosensor interface 130 can include redox cycle enzymes 134 attached to electrode 110. Redox cycle enzymes 134 may be directly or indirectly attached on electrode 110 as described further below. Primary analyte binding material 140 may be operably linked, directly or indirectly, in biosensor interface 130. A sample potentially containing analyte 144 is exposed to biosensor interface 130.

In one embodiment, if analyte 144 is present in the sample, then analyte 144 can bind to primary analyte binding material (i.e., capture analyte binding material) 140 and be immobilized on biosensor interface 130. The sample solution can then be replaced with another solution containing secondary analyte binding material 150 specific for analyte 144. Secondary analyte binding material 150 can be conjugated to reporter enzyme 154 such as a hydrolase. The secondary analyte binding material 150 conjugated to reporter enzyme 154 can bind to immobilized analyte 144. Another solution including substrate 160 can be added to biosensor interface 130. When substrate 160 is acted upon by reporter enzyme 154, trigger molecule 170 is formed that triggers the reaction of a redox cycle amplification pathway, leading to generation of an electric current.

In one embodiment, if analyte 144 is not present in a sample, then the above described steps do not lead to the generation of an electric current.

In various embodiments, trigger molecule 170 can diffuse to the redox cycle components of biosensor interface 130. Redox cycle components can include one or more enzymes 134, oxidized molecules 164 and reduced molecules 168. Oxidized molecules 164 and reduced molecules 168, for example, can be oxidized and reduced forms, respectively, of phenolic and quinone compounds. In one embodiment, trigger molecule 170 is oxidized molecule 164. In one embodiment, trigger molecule 170 is reduced molecule 168. In one embodiment, trigger molecule 170 is reacted by one or more enzymes 134 to form oxidized molecule 164. Whether trigger molecule 170 is oxidized molecule 164, reduced molecule 168, or reacted to form oxidized molecule 164 can depend on the identity of substrate 160 and reporter enzyme 154.

In one embodiment, reporter enzyme 154 may convert substrate 160, e.g. phenyl phosphate, into phenol. Phenol, as trigger molecule 170, enters the redox cycle. Enzyme 134, e.g. tyrosinase, can convert phenol to form catechol as reduced molecule 168. Catechol is then oxidized by enzyme(s) 134 to o-quinone as oxidized molecule 164 which in turn is reduced by electrode 110 to form catechol as reduced molecule 168, propagating a redox cycling loop between the oxidized and reduced forms of catechol. The current generated by the electrochemical reduction of oxidized molecule 164 can constitute electrical output of assay system 100. In various embodiments, trigger molecule 170 may be an intermediate that is then converted, by enzyme 134, to either oxidized molecule 164 or reduced molecule 168 to enter the redox cycle amplification pathway. In various embodiments, other components such as molecular oxygen, hydrogen peroxide or other electron acceptors may be included or added to the biosensor interface to propagate the redox cycle amplification pathway.

As discussed above, electrode 110 can include a variety of materials. In various embodiments, electrode compositions can include, but are not limited to, electrodes comprising certain metals and their oxides, including gold; copper; silver; lead; zinc; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃), ruthenium oxides; carbon (including glassy carbon electrodes, graphite and carbon paste) and a nonmetal substance, such as a composite material, for example, a carbon composite, and the like.

Electrodes can further contain or be in contact with and connected to electron conducting material, which includes, but is not limited to, a layer of the above-disclosed materials. In one embodiment, the electron conducting material can include gold. Conductive nanoparticles of gold or other metals may also be used to increase the effective surface area, which would increase the current generated by the biosensor.

In various embodiments, the electron conducting material can include carbon and/or materials that include carbon. Exfoliated nanographite, carbon nanotubes, fullerenes, and the like, may also be incorporated into an electrode. In one embodiment, the electron conducting material can include nanomaterials that include carboxyl functionalized carbon nanotubes. In one embodiment, the electron conducting materials can include graphene nanoparticles. Other electron conducting materials are also within the scope of this description. In one embodiment, the electron conducting molecules are located on the surface of the electrode.

In one embodiment, the biosensor device may be formed by using a layer-by-layer assembly approach for immobilizing a layer of primary analyte binding material on top of previously applied layers of one or more polymers and the enzymes of the redox cycle. In one embodiment, the polymer is a polysalt. In one embodiment, the polymer is any type of polyelectrolyte as defined herein.

In one embodiment, the biosensor has a response time on the order of seconds (e.g., less than about 100 seconds, or less than about 50 seconds, or less than about 20 seconds) and configured to reach a desired fraction, (such as >90%). In one embodiment, the biosensor has a dose-dependent sensor output that correlates with the amount of target analyte present in a given sample. The response time can be quicker if the concentration of the analyte in the sample is higher because less time would be required to get the analyte molecules to the primary antibodies and the secondary antibodies to the bound antigen.

In one embodiment, the biosensor device may be assembled without the use of a layer-by-layer assembly approach. Biosensor devices with or without layer by layer assembly are within the scope of this disclosure.

In various embodiments described herein, a variety of enzymes may be used to catalyze a redox cycle amplification pathway. Enzymes that can be used include enzymes that are known to oxidize phenolic compounds such as phenol, catechol and other phenolic compounds. These enzymes can include oxidoreductases that use oxygen as electron acceptor (E.C.1.10.3) and/or monophenol monooxygenase (E.C. 1.14.18.1). Enzymes of the redox cycle amplification pathway can also include enzymes that may not be directly involved in the redox cycle but provide components or catalyze reactions that enable the redox cycle to be initiated and/or propagated.

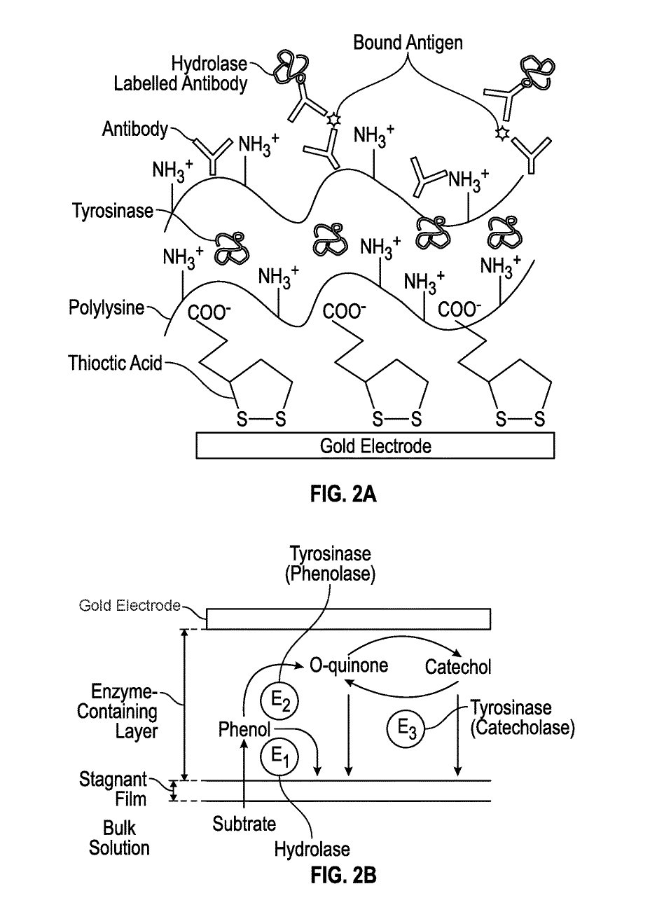

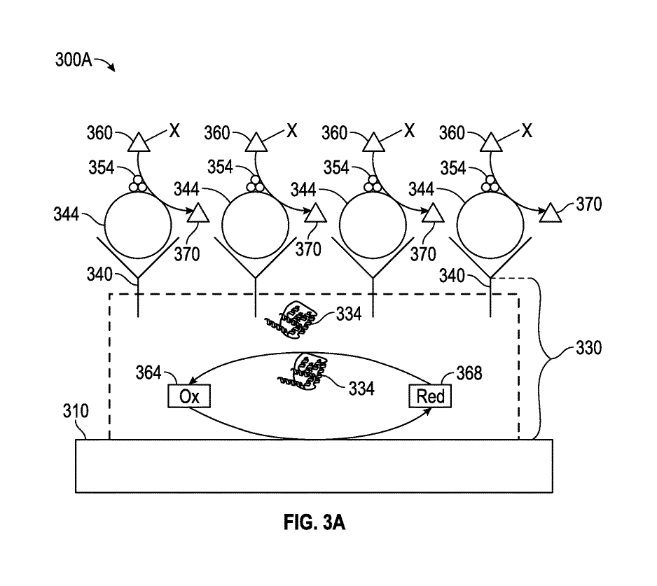

In various embodiments, a variety of oxidoreductases can be used as the redox enzymes and can include tyrosinase (also known as polyphenol oxidase), as well as catechol oxidase (E.C. 1.10.3.1) and ortho-aminophenol oxidase (E.C. 1.10.3.4). FIGS. 2A and 2B are schematic diagrams of one embodiment of an assay system that includes a tyrosinase biosensor interface in a biosensor device described herein. FIGS. 2A and 2B illustrate the use of a tyrosinase system in a sandwich mechanism when an analyte is present for catalyzing the redox cycle amplification pathway. As shown in FIG. 2B, a hydrolase enzyme can be used to convert the substrate into a product such as phenol that triggers a reaction in the redox cycle loop. The phenolic compound could be oxidized by tyrosinase to a quinone. The quinone would then be reduced back to a phenolic compound (e.g., catechol) by electron transfer from the electrode. The phenolic compound would then be re-oxidized by tyrosinase propagating a redox cycling loop between one or more quinones (e.g., o-quinone) and one or more phenolic compounds (e.g., catechol). The current generated by the electrochemical reduction of the quinone would constitute the biosensor's electrical output. An exemplary biosensor utilizing tyrosinase in a redox cycle amplification method is described in the '196 patent and in the '773 patent.

Briefly, tyrosinase is a copper-containing oxidase (Cache-Guerente, et al. (2001) Analytical Chemistry, 73:3206-3218; Forzani, et al. (2000) Analytical Chemistry, 72:5300-5307; all of which are herein incorporated by reference), which possesses two different activities, as illustrated in reaction (1).

phenol→catechol→o-quinone+H₂O (1)

The first step is referred to as the enzyme's phenolase activity (also known as cresolase activity) where phenol is hydroxylated by the aid of molecular oxygen to produce catechol. In the second step, known as the catecholase activity, the enzyme oxidizes catechol to o-quinone and is simultaneously oxidized by oxygen to its original form, with the production of water. The reaction product, o-quinone., is electrochemically active and can be reduced back to the catechol form at low applied electrical potential s, at the electrode as illustrated in reaction (2).

o-quinone+2H⁺+2e⁻→catechol (2)

These characteristics of the tyrosinase enzymes can be harnessed in the immunoassays described herein.

In one embodiment, the tyrosinase enzymes of the redox cycle are exemplified as being immobilized to the electrode by, but not limited to, thioctic acid and polylysine in FIG. 2A. Enzyme immobilizing agent can attach an enzyme to a biosensor of the present description. Examples of an enzyme immobilizing agents include, but are not limited to, any covalent bond forming compound, such as a cross-linking compound (e.g., via glutaraldehyde, thioctic acid, and the like), an entrapment compound, (e.g., polyacrylamide), a charged compound that can attached to the enzyme via ionic interactions (e.g., a positively charged polyelectrolyte, and the like, such as polylysine), a sensor-coupling molecule, and the like.

In one embodiment, the tyrosinase enzymes may be immobilized using other linkers such as glycine, and the like. Glycine, for example, may be used as a linker for biosensors having carbon electrodes. Biosensors with carbon electrodes are described, for example, in the '169 application.

In one embodiment, the tyrosinase enzymes may be attached directly to the electrode.

For the purposes of the present description attaching a molecule to an electrode may occur directly or indirectly, for example, a sensor-coupling molecule may be attached directly to an inorganic surface of an electrode whereas an enzyme may be attached indirectly to an electrode by attaching the enzyme to other molecules that in turn are attached to the electrode. Attachment of molecules to each other and molecules to inorganic surfaces may be accomplished by a variety of methods, such as by forming covalent bonds, electrostatic forces, entrapment, and the like.

In various embodiments, exemplified in FIG. 2A, a primary analyte binding material can be immobilized on the biosensor interface either directly or indirectly. The primary analyte binding material is generally operably attached to electrode and/or the enzymes that drive the redox cycle. By operably attached, it is meant that when the primary binding material is immobilized on the biosensor interface, it can still bind the target analyte.

In one embodiment, the primary analyte binding material can be immobilized directly to the electrode or to components in the biosensor interface.

In one embodiment, the primary analyte binding material may be immobilized to the biosensor interface through use of a linker, e.g. polylysine. Regardless of the immobilization method, the primary analyte binding material, when immobilized on the biosensor interface, is in a configuration that can bind the analyte of interest.

In various embodiments, the primary analyte binding material and the secondary analyte binding material can be any molecule configured to bind the target analyte. In one embodiment, the primary analyte binding material exhibits nonspecific binding. In one embodiment, the primary analyte binding material exhibits specific binding. In one embodiment, secondary analyte binding materials exhibit specific binding.

In various embodiments, the primary analyte binding material and/or the secondary analyte binding material are biological macromolecules, for example, antibodies or parts of antibodies. These molecules can also be receptors, ligands, polynucleotides, polypeptides, glycopeptides, lipoproteins, nucleoproteins, nucleic acid, aptamer, and the like.

In one embodiment, the primary analyte binding material and the secondary analyte binding material are both antibodies. In one embodiment, the primary analyte binding material is the same as the secondary analyte binding material. When the primary analyte binding material is the same as the secondary analyte binding material, they can bind the analyte at two different binding sites such that both the primary and secondary binding materials can simultaneously bind the analyte. In one embodiment, primary analyte binding material is different from the secondary analyte binding material and they bind the analyte at different binding sites.

In various embodiments described herein, the primary analyte binding material and the secondary analyte binding material are exemplified as being a primary antibody and a secondary antibody, respectively. However, analyte binding materials other than antibodies may be utilized and are included in scope of the present disclosure.

In various embodiments, the microorganism is selected from algae, bacteria, fungi (including lichens), Archaea, protozoa, viruses, and subviral agents.

In various embodiments, a variety of different primary antibodies can be included in the biosensor interface of the biosensor. In one embodiment, the specific primary antibody to be immobilized on the biosensor interface can be selected based on the target analyte of interest. In one embodiment, the target analyte is a fish pathogen or microorganism that causes disease in fish and the primary antibody is an antibody that can bind the fish pathogen of interest.

The biosensor devices with the electrode, the redox enzymes and the primary antibody can be assembled as described herein. These biosensors are compatible for use in a variety of immunoassay formats. These immunoassay formats are described further below and include, but are not limited to, sandwich assay formats, competitive displacement assay formats, and the like. The biosensor devices may also be compatible with the following components, including, but not limited to, lateral flow assays, vertical flow assays, printed electrodes, and the like.

In various embodiments, the biosensor immunoassays can include the biosensor devices and a secondary analyte binding composition that can be used or applied to the biosensor device after the application of a sample to be tested for the presence of an analyte. In one embodiment, the secondary analyte binding composition is provided as a separate solution so that it may be applied to the biosensor interface after the application of the sample to be tested. In one embodiment, the secondary analyte binding composition can include, for example, a secondary antibody conjugated to a reporter enzyme. The secondary antibody may include other components that are necessary for maintaining the function of the secondary antibody and/or the reporter enzyme. In the various embodiments, the secondary antibody can also vary depending on the specific immunoassay and the target analyte.

As indicated above for the primary antibody, if it is desirable to test a sample for the presence of a fish pathogen, the secondary antibody, in addition to the primary antibody, can also bind to the fish pathogen. Furthermore, the primary and the secondary antibodies can both bind to the target analyte at the same time. In other words, the binding site of the primary antibody on the analyte is different or sufficiently non-overlapping from the secondary antibody so that both the primary and the secondary antibody can bind the fish pathogen at the same time.

In various embodiments, the secondary antibody can be conjugated to a reporter enzyme. A variety of methods are known in the art to attach or couple the secondary binding material to the reporter enzyme and all are within the scope of this invention. A method to couple or conjugate binding material to reporter enzyme is described, for example, in Kricka, L J, “Selected Strategies for Improving Sensitivity and Reliability of Immunoassays”, Clinical Chemistry, Volume: 40, Issue: 3 Pages: 347-357, March 1994 and incorporated herein by reference.

In various embodiments, reporter enzymes can be hydrolases such as alkaline phosphatase, acetylcholinesterase (AchE), butyrylcholinesterase (i, e., BChE, BuChE, butylcholinesterase, pseudocholinesterase, plasma cholinesterase or liver cholinesterase), and neuropathic target esterase (NEST). In one embodiment shown in FIGS. 2A and 2B, ALP is used as the reporter enzyme but other enzymes can be used as the reporter enzyme and all are within the scope of the present disclosure. In one embodiment, ALP may be advantageous to use as a reporter enzyme since some antibodies conjugated to ALP may be commercially available.

In one embodiment of the chemical reaction exemplified in FIG. 2B, the substrate phenyl phosphate is used to initiate the chemical reaction in conjunction with ALP as the reporter enzyme to trigger the redox pathway. The ALP can cleave the phosphate group of phenyl phosphate resulting in phenol as the trigger molecule, which could then be oxidized as shown in the pathway in FIG. 2B to generate O-quinone which is in turn reduced by the electrode to generate an amplified electrical current.

In various embodiments, the biosensor immunoassays described herein can also include a substrate composition. The substrate composition can include a substrate that can be bound and catalyzed by the reporter enzyme. Furthermore, when the reporter enzyme binds and catalyzes the reaction with the substrate, the resultant product may be the trigger molecule. The trigger molecule can enter and activate the redox cycle in the biosensor interface resulting in propagation of the redox cycle and amplification of a signal that can correlate with the amount of analyte bound to the primary and secondary antibody.

In various embodiments, the redox enzymes can oxidize a variety of phenolic compounds and polyphenols to quinones. These phenolic compounds can have one or multiple hydroxyl groups and located at various positions on the aromatic ring. The phenolic compounds can also have other types of functional groups, such as nitro groups (NO₂), located at various locations relative to the hydroxyl group(s). Also, virtually any quinone formed by these reactions can be electrochemically reduced back to the phenolic compound at the electrode to initiate redox recycling for signal amplification. For these reasons, a wide variety of phenolic compounds, defined as compounds having one or more aromatic ring that contains one or more hydroxyl (OH) group, may be used.

In FIG. 2B, phenyl phosphate is exemplified as the substrate for the reporter enzyme, ALP. Although phenyl phosphate is shown as a substrate in conjunction with ALP as the reporter enzyme, other substrates can be used with ALP and/or with other reporter enzymes. In various embodiments, other substrates can include, for example, other phenolic substrates such as, phenyl valerate, phenyl acetate, and the like. In one embodiment, phenyl valerate can be used with NEST and/or butyrylcholinesterase as the reporter enzyme. In one embodiment, phenyl acetate can be used with acetylcholinesterase.

In one embodiment, the molecules that are reduced at the electrode in the redox cycle can include quinone molecules. In one embodiment, the quinone molecule reduced at the electrode is o-quinone. Other quinones may also be reduced at the electrode and all are within the scope of this disclosure.

In various embodiments, the reduced molecules that are oxidized by the redox enzymes can include phenol, catechol, and the like. Other phenolic reduced molecules may also be oxidized by the redox enzymes and all are within the scope of this disclosure.

In various embodiments, the presence of analytes in a variety of samples may be determined using the biosensor immunoassays and generally can be any type of liquid or solid sample. The samples may be biological samples, chemical samples, environmental samples, food samples, and the like.

In various embodiments, a biological sample may be obtained from any type of subject, as defined herein. In one embodiment, the subject is a human. In one embodiment, the subject is an animal. In one embodiment, the biological sample is fluid or tissue from a human or animal subject. In one embodiment, the biological sample may be obtained from food products and ingredients, including, but not limited to, dairy items, vegetables, meat and meat by-products, and waste, further including but not limited to, bodily fluids from any type of subject, including, but not limited to, blood, plasma, serum, urine, sweat, bile, tissue, cells, cerebrospinal fluid (CSF), fecal material, vaginal fluids, saliva, and the like, as well as proteins and nucleic add sequences. Other biological samples may also be analyzed and are all within the scope of the various embodiments described herein.

In various embodiments, an environmental sample can include any environmental material such as surface matter, soil, water, and industrial samples, as well as samples obtained from food and dairy processing instruments, apparatus, equipment, disposable, and non-disposable items. These examples are not to be construed as limiting the sample types applicable to the present disclosure.

In one embodiment, the sample with the target analyte may be used directly. In one embodiment, the sample with the target analyte may be diluted using a diluent. Diluent can be a variety of solutions and are generally known in the art. In one embodiment, the diluent is a saline solution.

In various embodiments, whether biological or environmental, a sample suspected of containing an analyte may first be subjected to enrichment means to create a concentrated sample. For example, “enrichment means” or “enrichment treatment,” contemplates isolating, purifying or partially purifying a particular protein, molecule, nucleic acid sequence of interest away from other proteins or chemicals. It is not intended that the present invention be limited only to one enrichment step or type of enrichment means. For example, it is within the scope of the present disclosure, following subjecting a sample to a conventional enrichment means, to subject the resultant preparation to further purification such that a purified protein of interest is produced. This purified protein may then be analyzed by devices and methods disclosed herein.

A variety of analytes can be detected using the methods and assay systems of the present description. In various embodiments, a target analyte can be a protein, peptide, nucleic acid, hapten, chemical, and the like. Analytes can also include therapeutic drugs, drugs of abuse, hormones, vitamins, glucose proteins, antibodies, steroids, bacteria or bacterial infection, fungi, viruses, parasites, components and products of bacteria, allergens, antigens, and the like. An analyte can also include derivatives or metabolites of the compound of interest.

In various embodiments, the bacteria are prokaryotic organisms. In one embodiment, the prokaryotic organisms are gram negative. In one embodiment, the prokaryotic organisms are gram positive. In one embodiment, the bacteria may be Mycoplasma, Chlamydia, Actinomyces, Streptomyces, and Rickettsia. In various embodiments, the bacteria form is selected from cocci, bacilli, spirochetes, spheroplasts, protoplasts, and the like.

In various embodiments, the analyte can be associated with a disease, for example, malaria, TB, and the like. In various embodiments, the analyte can be associated with a physiological or pathological condition, for example, pregnancy. Examples of analytes can include Cryptococcal antigen (CrAg), malarial antigen, Tuberculosis antigen, human chorionic gonadotropin (hCG), human luteinizing hormone (hLH), human follicle stimulating hormone (hFSF), prostate specific antigen (PSA), hepatitis B surface antigen, hepatitis B antibodies, HIV antigen, Streptococcus A, Staphylococcus bacteria, STDs, P. Falciparum, Fever panel, and the like. Other examples include pathogenic bacteria found in food such as the fish pathogen, Vibrio harveyi, and the like.

In various embodiments, the biosensor immunoassay can be used in a sandwich assay format. In the sandwich assay format, the biosensor interface initially contains no reporter enzyme, so the biosensor current produced can be essentially zero before exposure to the target analyte. When the biosensor interface is exposed to a sample containing the target analyte, the analyte can bind to the primary antibodies. Then, when the secondary antibodies are added, they can bind to the bound analyte, creating the sandwich architecture that can bind the reporter enzyme to the interfaces. The addition of the substrate can trigger the redox cycling loop, resulting in a biosensor current. Based on this mechanism, the higher the analyte concentration in the sample, the higher are the analyte and secondary antibody loading, and the higher the resulting biosensor signal. Because the initial background signal from the biosensor is essentially zero prior to analyte addition, even a small initial analyte concentration can provide a relatively large signal-to-background ratio. Thus, an advantage of the sandwich assay configuration can be that is has a relatively high sensitivity to detect low analyte concentrations.

Assay formats other than the sandwich format may also be used with the biosensor immunoassay. In various embodiments, the sandwich assay format can be effective, for example, for detecting cells (e.g., pathogens) and large molecules. Other assay formats may also be used for detecting cells and large molecules.

In various embodiments, small molecules may also be detected using the biosensor immunoassays. Eliciting antibodies against small molecules (analytes) in animals can be challenging. Even when antibodies are available that can bind a target small molecule, that molecule can be too small for two separate antibodies to bind simultaneously, as is required in the sandwich assay format.

In various embodiments for detecting small molecules, a competitive displacement assay format may be used. Antibodies that selectively bind to the target small analyte are obtained by using small analyte conjugates. These conjugates are produced by covalently binding the small analyte to another (conjugate) molecule that increases the small analyte's potency for stimulating the immune system. When the resulting conjugated small analyte is injected into an animal, the animal's immune system often produces antibodies against the conjugated small analyte that also selectively bind the original small analyte.

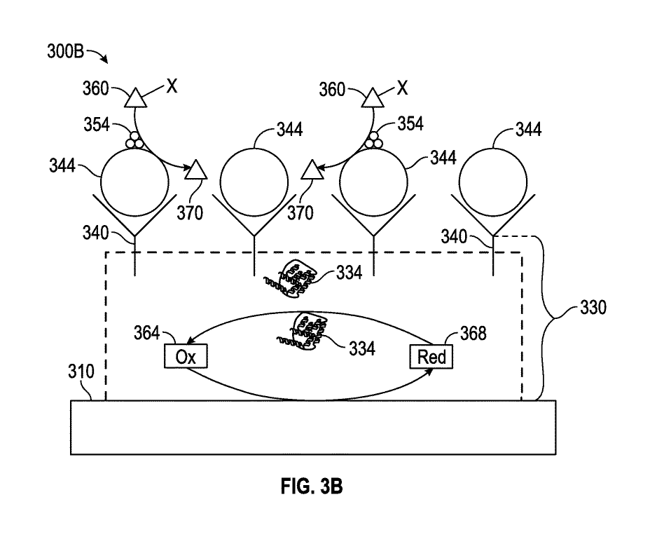

Using these type of antibodies, a competitive-displacement mechanism can be used to detect the small analyte. In one embodiment, FIG. 3A and FIG. 3B show a schematic of a competitive displacement assay. The identification numbers of the components common to FIG. 1 and in FIGS. 3A and 3B are correlated such that the components in FIGS. 3A and 3B are increased by 200 relative to FIG. 1. Electrode 310 of FIGS. 3A and 3B correlates with electrode 110 of FIG. 1 and so on.

In one embodiment of the competitive displacement approach, primary antibodies 340 are initially immobilized in biosensor interface 330 as shown in FIG. 3A. Then, biosensor interface 330 can be exposed to a solution containing a high concentration of hybrid reporter molecules that consist of small analyte 344 conjugated to reporter enzyme 354. Binding between primary antibodies 340 and the hybrid reporter molecules (analyte 344/reporter enzyme 354) is allowed to come to equilibrium, so that primary antibodies 340 are saturated with the hybrid reporter molecules (analyte 344/reporter enzyme 354). The high concentration of reporter enzyme 354 located on biosensor interface 330 can result in a maximum initial biosensor signal when the substrate composition is added before the exposure to the sample.

When biosensor interface 330 is contacted with a sample containing the target small analyte 344, analyte 344 (unconjugated) competes for primary antibody sites with the bound hybrid reporter molecules (analyte 344/reporter enzyme 354) and displaces some of the hybrid reporter molecules (analyte 344/reporter enzyme 354). As a result, the concentration of reporter enzymes 354 bound to biosensor interface 330 decreases, and thus the output current by electrode 310 can drop. In this way, the analyte concentration in the sample varies inversely with the current output by electrode 310 of biosensor 3000. The biosensor can be calibrated, so that the output current quantitatively indicates the analyte concentration in the sample.

The present description can also include methods for detecting the presence and concentration of a target analyte in a sample using the biosensor devices in immunoassays as described herein. The assay operating procedure may vary, depending on the enzymes used and whether a sandwich assay, a competitive-displacement assay, or some other assay formats are used.

In various embodiments for a sandwich assay format, the method can include exposing the biosensor interface to the sample solution to allow the analyte to bind to the immobilized primary antibodies. The next step can include replacing the sample solution with a solution containing the secondary analyte composition (secondary antibodies attached to the reporter enzyme). In one embodiment, replacing of the sample solution may not be necessary if the assay format is configured to absorb the liquid or have the liquid flow away from the biosensor interface. After binding of the secondary antibody/reporter enzyme molecules to generate the sandwich structure, the next step can include adding a substrate. Cleavage of the substrate by the reporter enzyme can generate a trigger molecule such as phenol or a phenolic compound, which can trigger the redox-cycling loop and result in generation of an electric current. The method can also include measuring the level of the current and determining the analyte concentration in the sample. The analyte concentration may be determined, for example, by calibrating with a sample containing a known analyte concentration.

In various embodiments for a competitive-displacement assay format, the method can include presaturating a biosensor interface with hybrid reporter enzymes/analyte molecules. Presaturating can allow the hybrid reporter enzymes/analyte molecule to bind to the primary analyte binding material in the biosensor interface. The method can include contacting a sample containing the analyte with the biosensor interface that was pre-saturated with hybrid reporter enzymes/analyte molecules. Analyte molecules in the sample can displace a fraction of the hybrid reporter enzyme/analyte molecules from the primary antibodies, thereby reducing the reporter enzyme concentration in the interface and reducing the biosensor's current output. After contacting the sample, a substrate composition containing the substrate can be added. The hybrid reporter enzymes/analyte molecules that have not been displaced by analyte in the sample can react with the substrate to form a trigger molecule as described herein. The method can also include measuring the level of the current and determining the analyte concentration in the sample. The analyte concentration may be determined, for example, by calibrating with a sample containing a known analyte concentration. In one embodiment, as the analyte concentration in a sample increases the signal from the assay can decrease since more of the analyte molecules in the sample can replace the hybrid reporter enzymes/analyte molecules bound to the primary analyte binding material.

In various embodiments, the competitive assay format may be advantageous for detection of small analyte molecules. Furthermore, this can involve fewer steps to measure the analyte concentration. In various embodiments, the sandwich assay format can be advantageous for exhibiting higher sensitivity and precision, because at low concentrations it measures small changes in current against a low background signal. The use of either competitive assay format or sandwich assay format for detecting large molecules, pathogens and/or small molecules is within the scope of the disclosure.

In various embodiments, the method can also include detecting multiple analytes. Multiple analytes can be detected by having a different primary antibody on a different electrode. The electrodes with the different primary antibodies can be formed into a biosensor array.

In various embodiments, biosensor devices with the biosensor interface may also be compatible with flow-through systems in which aliquots of liquid, e.g. sample, can be added to the biosensor interface and the liquid can be transported out of the reservoir after a desired amount of time via convective liquid flow driven by pumps, gravity, capillary forces, absorbents and/or membranes to control liquid flow rate.

In one embodiment, the flow-through systems can be compatible with a lateral flow immunoassay. In one embodiment, the flow-through system can be compatible with a vertical flow immunoassay. Absorbent materials can be used for this purpose in lateral flow immunoassays. Vertical flow immunoassays can also be employed in which the liquid flows downward through horizontal membrane layers. These horizontal membranes can be designed to be conductive to serve as both a membrane and an electrode. In one embodiment, the membranes can serve as immobilization supports for the immobilized biosensor components or reactants. The porosity and pore properties in the membranes can be customized to achieve the desired liquid flow rate through the membranes and thereby control the residence time of the liquids added to conduct the assay.

In various embodiments, the biosensor immunoassays may also be compatible with screen-printed electrode (SPE) arrays. An electrode may be constructed using inks for the deposition of electroconductive layers onto a support. Components of the biosensor interface may be attached to the electrode. The SPE arrays can be economically mass produced for applications including disposable POCT systems as described in “Screen-printed electrodes” Nascimento, V B; Angnes, L, ORCID QUIMICA NOVA, Volume: 21 Issue: 5 Pages: 614-629, September-October 1998, and incorporated herein by reference.

The amount of current generated in biosensor immunoassays can be measured by a variety of measuring devices. The measuring devices can be operably connected to the electrode so that the output of the device correlates with the current generated by the redox cycle at the electrode. In various embodiments, the measuring devices can include circuitry that can apply a desired voltage, and record, measure and provide electrical output, such as current, from the biosensor. The amount of current measured can then be correlated with the amount of analyte in a sample. These correlations can be determined by calibration methods, for example, by measuring biosensor currents for known concentrations of standard analyte solutions as understood by those skilled in the art. See, for example, “Implantable enzyme amperometric biosensors”, Kotanen, C N, et al., Biosensors & Bioelectronics, Vol. 35, Pp 14-26, DOI: 10.1016/j.bios.2012.03.016, May 15, 2012, which is hereby incorporated herein by reference.

In one embodiment, a specific voltage is applied to the biosensor device to measure the current. A variety of voltages may be applied but the amount of voltage applied can be sufficient to reduce the quinone molecules to the reduced phenolic molecules.

In one embodiment, the biosensor device is used in a voltammetry measurement. In one embodiment, the biosensor device is used in a potential step voltammetry measurement in which V1 can be a value that would not trigger an oxidation or reduction reaction and V2 might be a value that would cause that reaction to occur.

In one embodiment, a measuring device, such as a portable meter, comparable to a conventional glucose meter, may be used. Portable meters can detect and quantitate the current generated by the electrode. Portable meters useful in the various embodiments described herein are discussed in “Applications of commercial biosensors in clinical, food, environmental, and biothreat/biowarfare analyses,” Bahadir, E B et al., Analytical Biochemistry, Vol. 478, pp. 107-120, DOI: 10.1016/j.ab.2015.03.011, Jun. 1, 2015, which is incorporated herein by reference.

Other methods of detection may also be used for detecting the binding of the analyte to the biosensor devices and all are within the scope of this disclosure.

In one embodiment, an aquaculture pathogen can be detected using the biosensor immunoassays described herein. The aquaculture pathogen can be, for example, a Vibrio species such as Vibrio harveyi. Antibodies for these pathogens can be purchased from companies such as IBT Systems, Reutlingen, Germany, Sigma Aldrich, St. Louis, Mo. and ThermoFisher Scientific, Grand Island, N.Y. A secondary antibody conjugated to ALP can be applied to the biosensor interface after the contacting of a sample containing the fish pathogen. A substrate such as phenyl phosphate can be added and the current measured. In one embodiment, the assay can be a sandwich assay structure. In one embodiment, the assay can be a lateral flow assay format or a vertical flow assay format using a sandwich assay structure. Other formats and assay structures may also be used and all are within the scope of the disclosure.

In one embodiment, Tetrahydrocannabinol (THC) can be detected using the biosensor immunoassay disclosed herein. THC is a small molecule (molecular weight less than 2,000 Daltons. Antibodies to THC-conjugates can be used in immunoassays. In one embodiment, the assay structure can be a competitive displacement assay. This anti-THC antibody can bind strongly to either a THC molecule or an ALP-conjugated THC molecule. Anti-THC antibodies can be immobilized to the biosensor interface and preloaded with ALP-conjugated THC molecules.

In the presence of the triggering product, the high initial concentration of bound ALP molecules can generate a high current. However, upon exposure to a sample containing THC, the THC molecules in the sample can displace some of the THC-conjugated ALP molecules, in proportion to the THC concentration. In this way, the THC concentration in the sample can vary inversely with the biosensor current. The biosensor can be calibrated, so that the output amperage can provide a quantitative measure of the THC concentration.

This assay for testing THC can be used, for example, by any suitable end user, such as by law enforcement and/or a forensic professional as a roadside point of care testing (POCT) for driving-while-intoxicated (DWI) incidence. The results can be available to the officers within minutes, e.g., 10-15 minutes, as a numerical value that can be compared to the THC legal threshold level. In various embodiments, THC may be tested in samples of oral fluid, blood, urine, hair or sweat. THC is metabolized to 11-hydroxy-THC and carboxy-THC. Oral fluid (saliva) is a minimally invasive body fluid for illicit drug testing. In oral fluid, typically the parent compound, THC, is measured. Blood samples can be tested for both the parent compound and metabolites while urine can be tested for the metabolites.

In constructing the biosensor device described herein, in one embodiment, an optical assay based on the substrate tetramethylbenzidine (TMB), whose reaction product can be measured by absorbance at any suitable range can be used to confirm the formation of an immobilized biosensor interface. In one embodiment, the range is from about 600 to about 700 nm, such as from about 625 to about 675 nm, including any range there between, further including at least or no more than 650 nm. Other substrates whose reaction product can be measured by absorbance or other methods of detection may also be used.

In one embodiment, the substrate TMB can be added, and absorbance measured at a suitable wavelength to confirm the activity of the tyrosinase enzyme.

The formation of a functional sandwich ELISA interface can be confirmed using an ALP-conjugated goat anti-mouse-IgG as the secondary antibody conjugated to reporter enzyme. In one embodiment, the enzyme tyrosinase (redox enzymes) can be used to convert phenol to o-quinone, and 3-methyl-2-benzothiazolinone hydrazone (MBTH) to react with the quinone to produce a colored product that can be measured by absorbance at a lower suitable wavelength, such as from about 500 to about 510 nm, such as at least or no more than 505 nm. Other molecules to react with the quinone to produce a colored product may also be used.

Mouse IgG can be added to the biosensor interface to serve as the primary binding material in some embodiments. Mouse IgG can also serve as the analyte in one embodiment when used in combination with another primary antibody binding material that binds to mouse-IgG. Tyrosinase-conjugated goat anti-mouse-IgG can be added to, for example, high-throughput multiwell plates. Phenol and MBTH can be added and the absorbance measured at from about 500 to about 510 nm (e.g., about 505 nm) for testing the assembly of the secondary analyte binding material conjugated to the reporter enzyme.

In one embodiment, anti-mouse IgG can be attached to a working electrode as the primary antibody, and anti-mouse-IgG conjugated to tyrosinase can be used as the secondary antibody.

In the presence of the analyte, mouse IgG, a sandwich structure would be formed by the anti-mouse-IgG as the primary antibody, the mouse IgG as the analyte, and the tyrosinase-conjugated anti-mouse-IgG as the secondary antibody. In the presence of added phenol, the tyrosinase functionality can be determined by measuring a current resulting from redox cycling between catechol and o-quinone in a conventional electrochemical cell.

In another embodiment, anti-mouse-tyrosinase can be attached to the working electrode and ALP-conjugated anti-mouse-IgG can be used as the secondary antibody. In the presence of the analyte mouse IgG, a sandwich structure can be formed with anti-mouse-IgG as the primary antibody, the mouse IgG as the analyte, and the ALP-conjugated anti-mouse-IgG as the secondary antibody. In the presence of added substrates, phenyl phosphate and oxygen, the phenyl phosphate can be hydrolyzed by ALP to the trigger compound phenol, and then phenol can be oxidized to o-quinone by the tyrosinase. The biosensor functionality can be determined by measuring a current resulting from redox cycling between catechol and o-quinone in a conventional electrochemical cell.

The interface can be tested by titrating a known quantity of mouse-IgG and measuring the resulting current. The dose response curve can be measured by titrating with mouse-IgG and measuring the resulting current.

In one embodiment, a biosensor device comprising an electrically conductive electrode and a biosensor interface is provided. The biosensor interface comprising a primary antibody and one or more redox enzymes, wherein the one or more redox enzymes comprises tyrosinase and propagates a redox cycle by oxidizing a phenolic molecule to a quinone and wherein the electrode is configured to reduce the quinone to generate a reduced form of the phenolic molecule. The primary antibody is specific for a target analyte in a sample.

In various embodiments, the electrodes are selected from gold and carbon.

In one embodiment, the quinone is o-quinone and the phenolic molecule is catechol.

In one embodiment, the tyrosinase has phenolase activity and/or catecholase activity.

In one embodiment, the electrode is used in an amperometric measurement.

In one embodiment, an analyte detecting system is provided. The analyte detecting system comprises an electrically conductive electrode and a biosensor interface. The biosensor interface comprises a primary antibody and one or more redox enzymes, wherein the one or more redox enzymes comprises tyrosinase and propagates a redox cycle by oxidizing a phenolic molecule to a quinone and wherein the electrode is configured to reduce the quinone to generate a reduced form of the phenolic molecule. The primary antibody is specific for a target analyte in a sample.

In one embodiment, the system further comprising a detecting composition comprising a secondary antibody configured to bind the target analyte in the sample wherein the analyte binding sites of the secondary antibody and the primary antibody are different, the secondary antibody conjugated to a reporter enzyme.

In one embodiment, the system further comprises a substrate composition comprising a substrate wherein the reporter enzyme catalyzes the conversion of the substrate to a trigger molecule that triggers the redox cycle.

In one embodiment, the system further comprises a detecting composition comprising a target analyte conjugated to a reporter enzyme. In one embodiment, the system further comprises a substrate composition comprising a substrate wherein the reporter enzyme catalyzes the conversion of the substrate to a trigger molecule that triggers the redox cycle.

In one embodiment, the electrode a gold electrode. In one embodiment, the electrode is a carbon electrode.

In one embodiment, the quinone is o-quinone and the phenolic molecule is catechol.

In one embodiment, the tyrosinase has phenolase activity and/or catecholase activity.

In one embodiment, the reporter enzyme is ALP.

In one embodiment, the analyte is selected from a fish pathogen and/or THC.

In one embodiment, the sample is selected from a biological sample, environmental sample and/or chemical sample.

In one embodiment, a method for detecting a target analyte in a sample is provided. The method comprises providing a device comprising an electrically conductive electrode and a biosensor interface. The biosensor interface comprising a primary antibody and one or more redox enzymes, wherein the one or more redox enzymes comprises tyrosinase and propagates a redox cycle by oxidizing a phenolic molecule to a quinone and wherein the electrode is configured to reduce the quinone to generate a reduced form of the phenolic molecule. The primary antibody is specific for a target analyte in a sample. The method further comprises contacting the biosensor interface with a sample to be tested for the presence of a target analyte.

In one embodiment, the method further comprises adding a detecting composition to the primary antibody and any bound target analytes, the detecting composition comprising a secondary antibody configured to bind the target analyte in the sample wherein the analyte binding sites of the secondary antibody and the primary antibody are different, the secondary antibody conjugated to a reporter enzyme and contacting said device with a substrate of the reporter enzyme.

In one embodiment, the method further comprises adding a detecting composition to the primary antibody prior to the step of contacting of the sample, the detecting composition comprising the target analyte conjugated to a reporter enzyme and contacting said device with a substrate of the reporter enzyme.

In one embodiment, the electrode is a gold electrode. In one embodiment, the electrode is a carbon electrode.

In one embodiment, the quinone is o-quinone and the phenolic molecule is catechol.

In one embodiment, the tyrosinase has phenolase activity and/or catecholase activity.

In one embodiment, the reporter enzyme is ALP.

In one embodiment, the electrode is used in an amperometric measurement.

In one embodiment, the analyte is selected from a fish pathogen and THC.

In one embodiment, the sample is selected from a biological sample, environmental sample and chemical sample.

In one embodiment, the substrate is phenyl phosphate.

Although specific embodiments have been illustrated and described herein, any arrangements that achieve the same purpose, structure, or function may be substituted for the specific embodiments shown. For example, although the various embodiments have been described for detecting analytes in a sample, it is also expected that other assay structures, assay structure components and formats for assays can also be used for detecting analytes in samples. This application is intended to cover any adaptations or variations of the embodiments of the invention described herein, and these and other embodiments are within the scope of the following claims and their equivalents.

What is claimed is:

1. A biosensor device comprising:
an electrically conductive electrode; and
a biosensor interface comprising a primary antibody and one or more redox enzymes, wherein the one or more redox enzymes comprises tyrosinase and propagates a redox cycle by oxidizing a phenolic molecule to a quinone and wherein the electrode is configured to reduce the quinone to generate a reduced form of the phenolic molecule, the primary antibody specific for a target analyte in a sample.

2. The device of claim 1 wherein the electrode is selected from gold and carbon.

3. The device of claim 1 wherein the quinone is o-quinone and the phenolic molecule is catechol.

4. The device of claim 1 wherein the tyrosinase has phenolase activity and/or catecholase activity.

5. The device of claim 1 wherein the electrode is used in an amperometric measurement.

6. An analyte detecting system comprising:
an electrically conductive electrode; and
a biosensor interface comprising a primary antibody and one or more redox enzymes, wherein the one or more redox enzymes comprises tyrosinase and propagates a redox cycle by oxidizing a phenolic molecule to a quinone and wherein the electrode is configured to reduce the quinone to generate a reduced form of the phenolic molecule, the primary antibody specific for a target analyte in a sample.

7. The system of claim 6 further comprising a detecting composition comprising a secondary antibody configured to bind the target analyte in the sample wherein the analyte binding sites of the secondary antibody and the primary antibody are different, the secondary antibody conjugated to a reporter enzyme.

8. The system of claim 7 further comprising a substrate composition comprising a substrate wherein the reporter enzyme catalyzes the conversion of the substrate to a trigger molecule that triggers the redox cycle.

9. The system of claim 6 further comprising a detecting composition comprising a target analyte conjugated to a reporter enzyme.

10. The system of claim 9 further comprising a substrate composition comprising a substrate wherein the reporter enzyme catalyzes the conversion of the substrate to a trigger molecule that triggers the redox cycle.

11. The system of claim 6 wherein the electrode is selected from a gold electrode and a carbon electrode.

12. The system of claim 6 wherein the quinone is o-quinone and the phenolic molecule is catechol.

13. The system of claim 6 wherein the tyrosinase has phenolase activity and/or catecholase activity.

14. The system of claim 7 wherein the reporter enzyme is alkaline phosphatase.

15. The system of claim 9 wherein the reporter enzyme is alkaline phosphatase.

16. The system of claim 6 wherein the electrode is used in an amperometric measurement.

17. The system of claim 6 wherein the analyte is selected from a fish pathogen and tetrahydrocannabinol (THC).

18. The system of claim 6 wherein the sample is selected from a biological sample, environmental sample and chemical sample.

19. A method for detecting a target analyte in a sample comprising:
providing a device comprising an electrically conductive electrode and a biosensor interface, the biosensor interface comprising a primary antibody and one or more redox enzymes, wherein the one or more redox enzymes comprises tyrosinase and propagates a redox cycle by oxidizing a phenolic molecule to a quinone and wherein the electrode reduces the quinone to generate a reduced form of the phenolic molecule, the primary antibody specific for a target analyte in a sample; and
contacting the biosensor interface with a sample to be tested for the presence of a target analyte.

20. The method of claim 19 further comprising:
adding a detecting composition to the primary antibody and any bound target analytes, the detecting composition comprising a secondary antibody configured to bind the target analyte in the sample wherein the analyte binding sites of the secondary antibody and the primary antibody are different, the secondary antibody conjugated to a reporter enzyme; and
contacting said device with a substrate of the reporter enzyme.

21. The method of claim 19 further comprising:
adding a detecting composition to the primary antibody prior to the step of contacting of the sample, the detecting composition comprising the target analyte conjugated to a reporter enzyme; and
contacting said device with a substrate of the reporter enzyme.

22. The method of claim 19 wherein the electrode is selected from a gold electrode and a carbon electrode.

23. The method of claim 19 wherein the quinone is o-quinone and the phenolic molecule is catechol.

24. The method of claim 19 wherein the tyrosinase has phenolase activity and/or catecholase activity.

25. The method of claim 20 wherein the reporter enzyme is alkaline phosphatase and the substrate is a phenyl phosphate substrate.

26. The method of claim 19 wherein the electrode is used in an amperometric measurement.

27. The method of claim 19 wherein the analyte is selected from a fish pathogen and THC.

28. The method of claim 19 wherein the sample is selected from a biological sample, environmental sample and chemical sample.

29. The method of claim 21 wherein the reporter enzyme is alkaline phosphatase and the substrate is a phenyl phosphate substrate.
